(19) United States
(12) Patent Application Publication
(10) Pub. No.: US 2016/0235855 A1
(43) Pub. Date: Aug. 18, 2016

(54) STABLE AQUEOUS PARENTERAL PHARMACEUTICAL COMPOSITIONS OF INSULINOTROPIC PEPTIDES

(71) Applicant: SHANGHAI BENEMAE PHARMACEUTICAL CORPORATION, Shanghai (CN)

(72) Inventors: Chunlin XIONG, Shanghai (CN); Yunxia HE, Shanghai (CN); Yajun ZUO, Shanghai (CN); Gang YU, Shanghai (CN)

(21) Appl. No.: 15/045,068

(22) Filed: Feb. 16, 2016

Related U.S. Application Data
(63) Continuation of application No. PCT/CN2014/083370, filed on Jul. 31, 2014.

(30) Foreign Application Priority Data
Aug. 13, 2013 (CN) 201310351740.5

(51) Int. Cl. A61K 47/40; A61K 38/22; A61K 47/26; A61K 47/02; A61K 47/10; A61K 47/18; A61K 38/26; A61K 9/00

(52) U.S. Cl. CPC A61K 47/40; A61K 38/26; A61K 38/2278; A61K 9/0019; A61K 47/02; A61K 47/10; A61K 47/183; A61K 47/26

(57) ABSTRACT

Disclosed herein is an insulinotropic peptide multi-dose aqueous parenteral pharmaceutical composition and use thereof. A long-term storage formulation of the insulinotropic peptide can be obtained via the method of the present disclosure. The pharmaceutical composition of the present disclosure comprises: insulinotropic peptide, insulinotropic peptide analogue and derivative; pharmaceutically acceptable tonicity modifier (stabilizer); pharmaceutically acceptable preservative; and pharmaceutically acceptable dissolution enhancer and pharmaceutically acceptable buffer solution. The pharmaceutical composition of the insulinotropic peptide is used in the preparation of drugs for treating diabetes and adiposis.

### PRIORITY CLAIM

This application is a continuation of International Application No. PCT/CN2014/083370, filed Jul. 31, 2014, which claims priority to Chinese Patent Application No. 201310351740.5, filed Aug. 13, 2013, both of which are incorporated herein in their entirety.

### TECHNICAL FIELD

The present disclosure relates to an insulinotropic peptide multi-dose aqueous parenteral pharmaceutical composition and use thereof.

### BACKGROUND ART

Glucagon-like peptide 1 (also designated GLP-1) and Exendin-4 are both insulinotropic peptides, and have 53% identity in amino acid sequences thereof. Pharmacology has proven that both GLP-1 and Exendin-4 act on GLP-1 receptors of insulin-secreting β TC1 cells. This type of hormones can promote insulin secretion, and exert a glucose concentration-dependent hypoglycemic effect.

Similar to insulin, GLP-1 and Exendin are effective only when injected before meals. However, because a protein or polypeptide molecule is unstable, it cannot be developed to an oral pharmaceutical composition and must be used by injection. Even if a drug under development is in an injectable form, it tends to be a lyophilized injection powder that is inconvenient to use.

Instability of proteins and polypeptides includes two general aspects, i.e., physical and chemical aspects. Physical instability includes, for example, denaturation, surface adsorption, aggregation, precipitation, gelatination, and the like, and chemical instability includes, for example, hydrolysis, deamination, oxidization, racemization, isomerization, β-elimination, disulfide bond exchange, and the like. Such a series of unstabilizing factors will all change with the alteration in structures of the proteins or polypeptides, and therefore many protein or polypeptide drugs are produced by employing a freezing-drying method, such that the use of the formulation can meet the requirements for shelf life.

However, the production of lyophilized injection powders by employing the freezing-drying method suffers from many disadvantages: for example, high production cost, inconvenient for patients (the injection powder is in single doses, and prior to use each time, the patient needs to dissolve the injection powder with water, draw the mixture from a Penicillin bottle, and then injected the mixture), i.e., poor conformability, and therefore the market competitiveness is poor. Therefore, the development of multi-dose parenteral solutions not only provides convenience to patients, but also reduces the production cost, and thus has rather important significance for the improvement of market competitiveness.

Insulinotropic peptides, in particular GLP-1, have properties of these polypeptides, particularly physical instability, such as formation of gel, and therefore, in order to develop successfully multi-dose aqueous parenteral pharmaceutical compositions, these physical and chemical instability described above must be solved, to allow the compositions to achieve the pharmaceutically available period of validity.

In the development of multi-dose aqueous parenteral pharmaceutical compositions, adding a preservative into the pharmaceutical composition must be taken into consideration, so as to ensure that there is no microbial contamination during the storage duration and the usage period. However, most of preservatives are harmful to proteins or polypeptides, and interact with the proteins to make them unstable, leading to aggregation. For example, phenol preservatives, such as metacresol and phenol, cause human growth hormones to aggregate (Kirsch et al, 1993), phenol allows β-folds in insulin-type drugs to increase, and benzyl alcohol allows recombinant human interferon-γ to aggregate. Therefore, in the screening of pharmaceutical compositions, the relationship between the antimicrobial effect of the preservative and stability of the protein or polypeptide should be balanced. The trickiest difficulty in the development of parenteral solutions is to allow the formula to be able to be stored for 2 years or more at 4° C. after addition of a preservative. Many raw material drugs or stock solutions of proteins or polypeptides have no problem in storage for 2 years or more at 4° C., but have difficulty in meeting the requirements for shelf life after the addition of the preservative, just because the addition of the preservative will severely influence stability of the drug.

Dissolution enhancers selected for most proteins or polypeptides are surfactants and PEG. Surfactants are mostly Tween, Span, Poloxamer, Pluronic, Brij and the like. In addition to these substances, surfactants selected in the present disclosure further include propylene glycol and dextran. Propylene glycol and dextran have very good effects when used as GLP-1 dissolution enhancers.

Patent WO00/37098 filed by Brader, Mark, L. also mentions GLP-1 instability: physical stability is poor between pH 6.8 and 7.5, the formulation will become turbid at a pH value less than 8.0 after the addition of an preservative, and chemical stability is reduced when the pH value is greater than 8.8, therefore the suitable pH range is narrow, pH 8.2 to 8.8. A range claimed therein is 0.3 mg/mL to 0.65 mg/mL, and a particularly stable concentration is 0.5 mg/mL. They carried out the work employing synthetic GLP-1, which also indicates that GLP-1 is unstable indeed.

Whereas the present disclosure provides an aqueous parenteral pharmaceutical composition that allows GLP-1 to have greater stability, and the aqueous parenteral pharmaceutical composition has a GLP-1 concentration far higher than the drug concentration achievable by the prior art.

### SUMMARY OF THE INVENTION

In one aspect, the present disclosure provides an insulinotropic peptide multi-dose aqueous parenteral pharmaceutical composition that can be stored for a long term and have greater stability. The formulated aqueous parenteral pharmaceutical composition can meet the requirement for storage duration. The aqueous parenteral pharmaceutical composition comprises an insulinotropic peptide, a pharmaceutically acceptable isotonic agent, a pharmaceutically acceptable dissolution enhancer, a pharmaceutically acceptable preservative and a pharmaceutically acceptable buffer salt. Wherein, the aqueous parenteral pharmaceutical composition has a pH value of 3 to 5.

In the aqueous parenteral pharmaceutical composition of the present disclosure, the insulinotropic peptide is GLP-1, Exendin-4, a GLP-1 analogue, an Exendin-4 analogue, a GLP-1 derivative or an Exendin-4 derivative.

In the present application, the term “analogue” serves to denote such a peptide wherein one or more amino acid residues of the parent peptide have been substituted with other amino acid residues and/or wherein one or more amino acid residues of the parent peptide have been deleted and/or wherein one or more amino acid residues have been added into the parent peptide. This addition may be occurred at N-terminus or C-terminal or both, of the parent peptide. In general, an “analogue” is such a peptide wherein six or less amino acids of the parent peptide have been substituted and/or added and/or deleted, more preferably is such a peptide wherein three or less amino acids of the parent peptide have been substituted and/or added and/or deleted, and most preferably is such a peptide wherein one amino acid of the parent peptide have been substituted and/or added and/or deleted.

In the present application, the term “derivative” serves to denote such a peptide wherein one or more amino acid residues of the parent peptide have a substituent introduced therein, and typical variants of the substituent include amide, sugars, alkyl, acyl, ester, PEGylation and the like.

The insulinotropic peptide may be GLP-1, a GLP-1 analogue and a GLP-1 derivative.

Preferably, GLP-1 and the GLP-1 analogues have a sequence of:

6 10 20 30 37
X⁶HX⁸EGTFTSD VSSYLEX²²QAA X²⁶EFIAWLVX³⁴G X³⁶X³⁷

wherein X⁶ is: R or a deletion;

wherein X⁸ is: A, G or V;

X²² is: G or E;

X²⁶ is: K, R, Q or N;

X³⁴ is: K, R, Q or N;

X³⁶ is: R, R—NH₂, K or K—NH₂; and

X³⁷ is: G or a deletion.

The insulinotropic peptide may alternatively be Exendin-4, an Exendin-4 analogue and an Exendin-4 derivative.

Preferably, Exendin-4 and the Exendin-4 analogue have a sequence of:

X₁X₂X₃GTX₆X₇X₈X₉X₁₀ SKQX₁₄EEEAVX₂₀ LX₂₂X₂₃X₂₄X₂₅LKNGG
X₃₁X₃₂X₃₃X₃₄X₃₅X₃₆X₃₇X₃₈X₃₉

wherein, X₁ is H, R or Y;

X₂ is S, G, A or T;

X₃ is D or E;

X₆ is F or Y;

X₇ is T, Y or S;

X₈ is S or Y;

X₉ is D or E;

X₁₀ is L or I;

X₁₄ is L, I, V or M;

X₂₀ is R or K;

X₂₂ is F or Y;

X₂₃ is I, V, L or M;

X₂₄ is E or D;

X₂₅ is W, For Y;

X₃₁ is P or a deletion;

X₃₂ is S or a deletion;

X₃₃ is S or a deletion;

X₃₄ is G or a deletion;

X₃₅ is A or a deletion;

X₃₆ is P or a deletion;

X₃₇ is P or a deletion;

X₃₈ is P or a deletion; and

X₃₉ is S, R or a deletion.

For example, Exendin-4 and the Exendin-4 analogue may have a sequence of:

HGEGTFTSDL SKQMEEEAVR LFIEWLKNGG PSSGAPPPS

As is well known, the insulinotropic peptide has poor stability, and it is very difficult for the aqueous solution thereof to get through its storage duration (2 years at 2 to 8° C.). However, the present disclosure has developed a pharmaceutical composition capable of allowing the insulinotropic peptide, the analogue and the derivative to be stable, and allow them to meet the requirement for the storage duration. For example, in the formulation of the present disclosure, the GLP-1 concentration is up to 2 mg/ml, and in the event that an preservative is added, it can be stored for 2 years at 4° C.

Concentration is a factor that influences stability. If a drug molecule has good stability, its steady concentration is high, and on the contrary, its steady concentration is low. For a certain particular drug molecule, the drug has greater stability at a low concentration than that at a higher concentration.

In the pharmaceutical composition of the present disclosure, the insulinotropic peptide has a concentration of about 0.1 to 20 mg/mL, more preferably about 0.2 to 10 mg/mL, more preferably about 0.05 to 0.5 mg/mL, more preferably about 0.5 to 5 mg/mL, more preferably about 1 to 5 mg/mL, and more preferably about 2 to 4 mg/mL. In the present application, “about” refers to the difference from a stated numerical value in a range of ±10%.

Another factor that that plays an important role in stability is maintenance of the pH value of the pharmaceutical composition, and in particular, it is found in the present disclosure that maintenance of the pH value at about 3.5 to 4.0 is very good, and GLP-1 can maintain good stability within this range. Also, it is found that the pH value at which stability is kept has a very narrow range. The drug is very unstable at a pH value between about 4.5 and 6.5, and turbidity or precipitation will be occurred so long as the GLP-1 drug molecules are shifted into this pH range. When the pH value is lower than about 3.5, acid hydrolysis will be occurred, which is unstable likewise. Also, the injection formulation requires that the pH value should not be lower than about 3.0, most preferably not be lower than about 4.0, and a pH value lower than about 3.5 or lower is unfavorable for animal and human bodies.

The pharmaceutical composition of the present disclosure has a pH value of about 3.5 to 5.0, more preferably about 3.5 to 4.5, more preferably about 3.6 to 4.2, more preferably about 3.6 to 4.0, and more preferably about 3.6 to 3.9. At this pH, stability of GLP-1 comes up to what is expected, and can be stored for 2 years or more at 2 to 8° C.

In the process of formulating the GLP-1 pharmaceutical composition formulation, it is generally required to add a buffer salt to maintain pH of the pharmaceutical composition. In addition, the kind of the buffer will also influence the stability of GLP-1. Phosphate has poor stability, and the buffer salt should be able to provide a buffer salt within this pH range, hence histidine-HCl, sodium acetate-acetic acid, glycine-HCl, disodium hydrogen phosphate-citric acid, sodium hydroxide-citric acid, sodium citrate-citric acid, succinate-succinic acid, lactate-lactic acid, glutaminate-glutamic acid, malate-malic acid, benzoate-benzoic acid, tartrate-tartaric acid and the like may be employed. This buffer salt must be a pharmaceutically acceptable buffer salt, i.e., it has no adverse effect on GLP-1, and has pharmacology and toxicology that meet the requirements. The buffer salt is preferably histidine and sodium acetate-acetic acid, and most preferably sodium acetate-acetic acid.

Concentration of the buffer salt has a very great influence on the GLP-1 polypeptide. GLP-1 is highly sensible to the salt and is extremely unstable to the salt at a high concentration. Concentration of the buffer salt selected in the present disclosure is about 2 to 200 mmol/L, more preferably about 5 to 200 mmol/L, more preferably about 5 to 50 mmol/L, more preferably about 5 to 20 mmol/L, and most preferably about 7.5 to 10 mmol/L.

For a multi-dose parenteral solution, the preservative is an essential ingredient of the pharmaceutical composition formulation. Preservative refers to a natural or synthetic chemical ingredient, for adding into food, drugs, pigments, biological specimens and the like, to delay decomposition caused by microbial growth or chemical changes, and thereby to prolong shelf life of the food, drugs, pigments, biological specimens and the like. If no preservative is added into the polypeptide drug, it is extremely difficult to meet the quality control requirements of microbes for multiple administrations. Usage of the preservative has much to do with the kind of the preservative, the pH value of the pharmaceutical composition, the packaging material and the sealing material. Preservatives of the types such as Nipagin and benzoic acid have high preservative efficacy at an acidic condition, and reduced efficacy at an alkaline condition. Various preservatives all have effective antimicrobial concentrations thereof, and the concentration in use should not be lower than these concentrations. Also, the preservative should be used in an amount that is not too high, to prevent from doing harm to human bodies. Preservatives that may be used in drugs may influence stability of the polypeptide. Generally, phenols are used in the pharmaceutical composition as preservatives, but phenol preservatives have severe influences on the stability of polypeptides, and GLP-1 as well. Therefore, it is a difficult problem that must be solved to carefully select an preservative for a GLP-1 drug solution, which not only has an preservative effect, but also will not notably influence the stability of GLP-1, i.e., after employment, it allows the pharmaceutical composition to be able to meet the requirements for storage duration. The present disclosure has successfully solved this difficult problem.

When the insulinotropic peptide is GLP-1, a GLP-1 analogue or a GLP-1 derivative, the preservative may be phenol, benzyl alcohol, methyl p-hydroxybenzoate, ethyl p-hydroxybenzoate, propyl p-hydroxybenzoate, butyl p-hydroxybenzoate, chlorobutanol, 2-phenoxyethanol, 2-phenethyl alcohol, benzalkonium chloride (bromide), merthiolate or any combinations thereof. When the insulinotropic peptide is Exendin-4, an Exendin-4 analogue or an Exendin-4 derivative, the preservative may be phenol, metacresol, benzyl alcohol, methyl p-hydroxybenzoate, ethyl p-hydroxybenzoate, propyl p-hydroxybenzoate, butyl p-hydroxybenzoate, chlorobutanol, 2-phenoxyethanol, 2-phenethyl alcohol, benzalkonium chloride (bromide), merthiolate or any combinations thereof. For a GLP-1 pharmaceutical composition, preferably benzyl alcohol, methyl p-hydroxybenzoate, ethyl p-hydroxybenzoate, propyl p-hydroxybenzoate and phenol are used, more preferably benzyl alcohol, phenol, or two of the above are used in combination. For an Exendin-4 pharmaceutical composition, preferably metacresol, benzyl alcohol, methyl p-hydroxybenzoate, ethyl p-hydroxybenzoate, propyl p-hydroxybenzoate and phenol are used, more preferably metacresol, benzyl alcohol, phenol, or two of the above are used in combination.

Concentration of the preservative is also a factor to be taken into consideration. Different kinds of preservatives may have different antimicrobial concentrations in use. If metacresol or phenol is selected, the concentration in use is about 1 mg/mL to 10 mg/mL, more preferably about 1 mg/mL to 5 mg/mL, and most preferably about 1.5 mg/mL to 3 mg/mL. If benzyl alcohol is selected, the concentration in use is about 5 mg/mL to 20 mg/mL, more preferably about 5 mg/mL to 10 mg/mL, and most preferably about 7.5 mg/mL to 10 mg/mL.

In the formulation of the pharmaceutical composition, an isotonic agent should be selected carefully to allow the pharmaceutical composition to have an tonicity that is close to the human tonicity. In addition, many isotonic agents function as stabilizers at the same time. Not only an intrinsic tonicity of the isotonic agent, but also influences of other ingredients in the pharmaceutical composition on the overall tonicity of the composition should be taken into consideration in the selection of concentration of the isotonic agent. Isotonic agents employed in the present disclosure include polyols, for example, mannitol, sorbitol, inositol, xylitol, glycerin, propylene glycol and the like; sodium chloride; sugar, sucrose, trehalose, lactose, fructose and glucose and the like, and mannitol, glycerin and sorbitol are used preferentially, most preferably mannitol.

When polyol or sugar is used as the isotonic agent, it is used at a concentration of about 10 mg/mL to 100 mg/mL, and more preferably about 30 mg/mL to 50 mg/mL. When sodium chloride is used as the isotonic agent, it is used at a concentration of about 1 to 30 mg/mL, more preferably about 1 to 15 mg/mL, more preferably about 5 mg/mL to 15 mg/mL, and more preferably about 7 mg/mL to 9 mg/mL.

Because GLP-1 has strong hydrophobicity and is susceptible to self-association into macromolecular aggregates or generation of gels, a dissolution enhancer has a very good effect on the dissolution of GLP-1. Dissolution enhancers employed in the present disclosure include Tween 20, Tween 40, Tween 80, Span 20, Span 40, Span 80, Poloxamer 188, Pluronic F68, Brij 35, dextran, PEG 400, PEG 1000, PEG 1500, PEG 2000, propylene glycol, and the like, and propylene glycol and PEG 400 are used preferentially, most preferably propylene glycol. The dissolution enhancer is used at a concentration of about 0.1 mg/mL to 10 mg/mL, and preferably about 0.2 mg/mL to 5 mg/mL.

In another aspect, the present disclosure further provides use of the aqueous parenteral pharmaceutical composition formulated in the present disclosure, in particular, use thereof in the preparation of drugs for treating diabetes and adiposis.

The aqueous parenteral pharmaceutical composition of the present disclosure overcomes the problem with a parenteral solution in the prior art of difficulty in meeting the requirements for shelf life after the addition of an preservative, and is still able to be stored for 2 years at 4° C. in the event that an preservative is added therein at a concentration up to 2 mg/ml.

The present disclosure is further set forth below in conjunction with particular embodiments. However, the present disclosure is not limited to these particular embodiments or examples.

### EXAMPLE 1

### Influence of pH Value and Ionic Strength on Stability of GLP-1

GLP-1 lyophilized powders were taken, dissolved with a 0.01 M sodium acetate-acetic acid buffer to 10 mg/mL, and then GLP-1 was replaced into buffers at various pH values through dialysis or through a G25 chromatographic method. Each buffer was also designed to have 4 salt concentrations. Samples collected were quantified by a HPLC method, and then GLP-1 concentration was adjusted to 4 mg/mL, followed by addition of an adjuvant to a required concentration. The final concentration of GLP-1 was 2 mg/mL. The kinds of the buffers and designs of pharmaceutical compositions are as shown in Table 1.

TABLE 1
Observation of influences of pH and salt concentration
on physical stability of GLP-1

| | | Replacement | NaCl con- | |
|---|---|---|---|---|
| Type of the | | methods of the | centration | Situations of samples |
| buffers | pH | buffers | (mmol/L) | after preparation |
| 20 mmol/L | 3.0 | Sephadex G25 | 0 | Clear and transparent |
| NaAC-HAC | | | 20 | Clear and transparent |
| | | | 150 | Clear and transparent |
| | | | 500 | Clear and transparent |
| | 3.5 | Sephadex G25 | 0 | Clear and transparent |
| | | | 20 | Clear and transparent |
| | | | 150 | Clear and transparent |
| | | | 500 | Turbid |
| | 4.0 | Sephadex G25 | 0 | Clear and transparent |
| | | | 20 | Clear and transparent |
| | | | 150 | Clear and transparent |
| | | | 500 | Turbid |
| | 4.5 | Dialysis | 0 | Difficult to filter |
| | | | 20 | Difficult to filter |
| | | | 150 | Difficult to filter |
| | | | 500 | Difficult to filter, |
| | | | | turbid |
| | 5.0 | Dialysis | 0 | Turbid or precipitated |
| | | | 20 | |
| | | | 150 | |
| | | | 500 | |
| 20 mmol/L | 5.5 | Dialysis | 0 | Turbid or precipitated |
| citrate | | | 20 | |
| sodium- | | | 150 | |
| citric acid | | | 500 | |
| | 6.0 | Sephadex G25 | 0 | Precipitate sticking |
| | | | | to the wall |
| | | | 20 | Precipitate sticking |
| | | | | to the wall |
| | | | 150 | Precipitate sticking |
| | | | | to the wall |
| | | | 500 | Precipitate sticking |
| | | | | to the wall |
| | 6.5 | Sephadex G25 | 0 | Turbid |
| | | | 20 | Turbid |
| | | | 150 | Turbid |
| | | | 500 | Turbid |
| 20 mmol/L | 7.0 | Sephadex G25 | 0 | Clear and transparent |
| Na₂HPO₄₋ | | | 20 | Clear and transparent |
| | | | 150 | Clear and transparent |
| | | | 500 | Clear and transparent |
| NaH₂PO₄ | 7.5 | Sephadex G25 | 0 | Clear and transparent |
| | | | 20 | Clear and transparent |
| | | | 150 | Clear and transparent |
| | | | 500 | Clear and transparent |

Samples were placed at two temperatures, i.e., 25° C. and 35° C., for treatment, and taken out for detection at 8 days.

An investigation method for physical stability: before determination, the samples were observed for appearance with the naked eye. If there was evident precipitation or turbidity, no further determination would be carried out. If the samples had no abnormal phenomena observable with the naked eye, the samples were taken and subjected to determination of absorption values at 360 nm and HPLC detection, wherein the former investigated the physical stability, and the latter investigated the chemical stability.

Absorption values determined at a wavelength of 360 nm were compared, so as to compare differences in physical stability among the samples. A higher absorption value indicated poorer physical stability.

An investigation method for chemical stability: a test sample was taken, and analyzed on a C18 column (3.5 μm, 300 Å, φ 4.6×50 mm). An analytical method: mobile phase A was 0.1% trifluoroacetic acid, B was the A phase with 80% acetonitrile added therein, the detection wavelength was 280 nm, 60% A and 40% B were balanced for 3 min, and the sample amount was 2 to 5 μL. Elution was performed with linear gradient for 8 min with 40% to 53% of the B phase and 60% to 47% of the A phase. Peak area was calculated using the normalization method. A standard sample was determined with the same method. The peptide concentration in the sample was calculated by comparison with the standard sample, and compared with the determination result at day 0, to calculate the retention of the peptide content in the sample. A higher retention of the peptide content indicated better chemical stability.

Stability results of GLP-1 in solutions at various pH values are as follows.

TABLE 2
Influences of pH and salt concentration on stability of GLP-1

| | | | | Results of HPLC analysis, | |
|---|---|---|---|---|---|
| | NaCl | Absorption values | | retention of the peptide | |
| | concentration | at 360 nm | | content (%) | |
| pH | (mg/mL) | 35° C., 8 days | 45° C., 8 days | 35° C., 8 days | 45° C., 8 days |
| 3.0 | 0 | 0.0706 | 0.0844 | 95.46 | 87.19 |
| | 1.17 | 0.0716 | 0.0724 | 93.69 | 87.93 |
| | 8.77 | 0.1697 | 0.1812 | 95.03 | 87.86 |
| | 29.22 | 0.2052 | 0.2417 | 96.42 | 91.16 |
| 3.5 | 0 | 0.0684 | 0.0689 | 94.73 | 88.66 |
| | 1.17 | 0.0694 | 0.0823 | 96.92 | 92.54 |
| | 8.77 | 0.1882 | 0.1780 | 98.06 | 92.90 |
| | 29.22 | Samples became turbid by placement for a period of | | | |
| | | time after formulation. | | | |
| 4.0 | 0 | 0.0686 | 0.0843 | 96.07 | 92.77 |
| | 1.17 | 0.1354 | 0.1448 | 97.43 | 93.76 |
| | 8.77 | 0.1907 | 0.1974 | 98.73 | Turbid |
| | 29.22 | Samples became turbid after formulation. | | | |
| 4.5 | 0 | 0.1326 | 0.1370 | 97.23 | 92.45 |
| | 1.17 | 0.1655 | 0.1814 | 95.29 | Turbid |
| | 8.77 | 1.4096 | 1.3635 | Turbid | Turbid |
| | 29.22 | Samples became turbid after formulation. | | | |
| 5.0 | 0 | Precipitate was generated while GLP-1 was | | | |
| | 1.17 | prepared. | | | |
| | 8.77 | | | | |
| | 29.22 | | | | |
| 5.5 | 0 | Precipitate was generated while GLP-1 was | | | |
| | 1.17 | prepared. | | | |
| | 8.77 | | | | |
| | 29.22 | | | | |
| 6.0 | 0 | Precipitate was generated after formulation and | | | |
| | 1.17 | placement of the sample. | | | |
| | 8.77 | | | | |
| | 29.22 | | | | |
| 6.5 | 0 | Turbidity was generated after formulation and | | | |
| | 1.17 | placement of the sample. | | | |
| | 8.77 | | | | |
| | 29.22 | | | | |
| 7.0 | 0 | 0.0813 | 0.0828 | 94.30 | 88.83 |
| | 1.17 | 0.0864 | 0.0865 | 93.35 | 90.43 |
| | 8.77 | 0.0824 | 0.0841 | 94.34 | 92.79 |
| | 29.22 | 0.0850 | 0.0862 | 95.83 | 95.37 |
| 7.5 | 0 | 0.0807 | 0.0859 | 90.89 | |
| | 1.17 | 0.0799 | 0.0937 | 91.88 | 85.88 |
| | 8.77 | 0.0846 | 0.0902 | 92.15 | 87.48 |
| | 29.22 | 0.0861 | 0.0942 | 93.74 | 88.04 |

Note:
an absorption value of the control background color was 0 to 0.1, and opalescence or turbidity could be seen with the naked eye when it was greater than 0.12.

Conclusions: when GLP-1 was at pH 5.0 to 6.5, turbidity or precipitation was occurred while the GLP-1 sample was prepared, indicating that GLP-1 is unstable at these pH values; whereas when pH was 4.5, the sample prepared was clear and transparent, but turbidity was occurred in case of high salt concentration when formulated as a pharmaceutical composition and then subjected to heating treatment. At pH 3.5 and pH 4.0, turbidity was occurred in the sample in the presence of 0.5 M NaCl, and no turbidity or precipitation was seen only at pH 3.0. As can also be seen from the absorption values determined at 360 nm, the absorption value was increased with the increasing pH value between pH 3.0 and 4.5, indicating that the physical stability was decreased. At the same pH value, the absorption value was also increased with the increasing NaCl salt concentration, indicating that the salt decreased the physical stability. However, the HPLC analysis was then contrary, the retention of the peptide content was increased with the increasing pH value, indicating that a superacidic condition is unfavorable for the chemical stability of GLP-1. Whereas at the same pH value, the retention of the peptide content was increased with the increasing salt concentration, possibly due to a certain inhibitory effect of the salt on GLP-1 adsorption. At pH 7.0 and 7.5, the physical stability was all normal, but the retention of the peptide content was significantly lower as compared with the samples at pH 3.0 to 4.5, and therefore the chemical stability was poor at pH 7.0 to 7.5.

The following table shows the physical stability and chemical stability of the pharmaceutical composition when pH of the GLP-1 pharmaceutical composition ranged from 3.6 to 4.2 and the sodium chloride salt concentration was below 20 mmol/L (1.17 mg/mL). A monitoring method for physical stability of the pH value was the fluorescence value, that is, thioflavine T was added into the sample at a final concentration of 5 μmol/L, then the fluorescence absorption value was determined (at an excitation wavelength of 435 nm, and an emission wavelength of 485 nm). The higher the absorption value is, the severer the gelatination phenomena of the sample is, and the poorer the physical stability is.

TABLE 3
Influence of the pH value on stability of GLP-1

| | Results of HPLC analysis, | | | |
|---|---|---|---|---|
| | retention of the peptide content % | | Fluorescence value | |
| pH | 25° C., 30 days | 35° C., 30 days | 25° C., 35 days | 35° C., 35 days |
| 3.6 | 93.04 | 86.25 | 8.49 | 9.95 |
| 3.7 | 95.15 | 86.11 | 9.11 | 14.55 |
| 3.8 | 97.10 | 89.48 | 11.01 | 31.58 |
| 3.9 | 98.08 | 90.36 | 26.46 | 78.27 |
| 4.0 | 97.85 | 91.80 | 59.32 | 113.18 |
| 4.1 | 98.90 | 91.35 | 139.48 | 157.00 |
| 4.2 | 98.24 | 93.96 | N.D | N.D |

As can be seen from the results in Table 3, between a range from pH 3.6 to 4.2, the lower the pH value is, the better the physical stability is, and the poorer the chemical stability is, and on the contrary, the higher the pH value is, the better the chemical stability is, and the poorer the physical stability is. Therefore, the pH range at which GLP-1 is stable is supposed to be a result of comprehensive consideration of both physical and chemical stability.

### EXAMPLE 2

### Preparation of the Formulation Pharmaceutical Composition

20 mL of 4 mg/mL GLP-1 peptide (in a 20 mmol/L buffer, pH 3.5 to 4.5) was mixed with 20 mL of 80 mg/mL mannitol-5.2 mg/mL phenol. The mixture was adjusted to pH 3.5-4.5 with NaOH or acetic acid, filtered through a 0.22 μm filter membrane, and dispensed into 2 mL Penicillin bottles. Each of the components was:

| | | |
|---|---|---|
| GLP-1 | 2 | mg/mL |
| Mannitol | 40 | mg/mL |
| Phenol | 2.6 | mg/mL |
| NaAC-HAC | 10 | mmol/L |

| | |
|---|---|
| pH | 3.5 to 4.5 |

The samples dispensed were placed at 25° C. and 35° C. respectively. Samples were taken at different times for inspection and analysis, to investigate physical and chemical stability.

### EXAMPLE 3

### Influences of Buffer Systems and Antimicrobial Agents on the Physical Stability of GLP-1

The GLP-1 solution (referred to as a stock solution) that had been replaced into different buffer systems (the buffers had a concentration 2 times that of the final pharmaceutical composition) was diluted with a buffer to 4 mg/mL, and an equal volume of a concentrated stock adjuvant solution with a 2-time final concentration was added therein. The solutions were mixed uniformly, filtered through a 0.22 μm filter membrane, dispensed into 2 mL Penicillin bottles, and placed at different temperatures for investigation. A series of sampling time points were arranged. After sampling, the samples were firstly observed with the naked eye for appearance. If evident turbidity or precipitation was occurred, the sample was considered as disqualified as for physical stability, and would not be subjected to the next step of HPLC analysis.

Designs and results are as shown in Table 4:

TABLE 4
Influence of composition of the pharmaceutical composition on the physical stability of GLP-1

| Pharmaceutical | Composition | Appearance | |
|---|---|---|---|
| composition No. | (GLP-1 final concentration 2 mg/mL) | after preparatio [symbol] | 25° C., 14 days |
| 1 | 10 mmol/L NaAC-HAC pH 3.5, 40 mg/mL | Turbid | Turbid |
| | mannitol, 3 mg/mL metacresol | | |
| 2 | 10 mmol/L NaAC-HAC pH 3.5, 45 mg/mL | Turbid | Turbid |
| | mannitol, 3 mg/mL metacresol | | |
| 3 | 10 mmol/L NaAC-HAC pH 3.5, 50 mg/mL | Turbid | Turbid |
| | mannitol, 3 mg/mL metacresol | | |
| 4 | 10 mmol/L NaAC-HAC pH 3.5, 55 mg/mL | Turbid | Turbid |
| | mannitol, 3 mg/mL metacresol | | |
| 5 | 10 mmol/L NaAC-HAC pH 3.5, 40 mg/mL | Transparent and | Transparent and |
| | mannitol, 2 mg/mL phenol | clear | clear |
| 6 | 10 mmol/L NaAC-HAC pH 3.5, 45 mg/mL | Transparent and | Transparent and |
| | mannitol, 2 mg/mL phenol | clear | clear |
| 7 | 10 mmol/L NaAC-HAC pH 3.5, 50 mg/mL | Transparent and | Transparent and |
| | mannitol, 2 mg/mL phenol | clear | clear |
| 8 | 10 mmol/L NaAC-HAC pH 3.5, 55 mg/mL | Transparent and | Transparent and |
| | mannitol, 2 mg/mL phenol | clear | clear |
| 9 | 10 mmol/L NaAC-HAC pH 3.5, 10 mg/mL | Turbid | Turbid |
| | glycerin, 3 mg/mL metacresol | | |
| 10 | 10 mmol/L NaAC-HAC pH 3.5, 1.50 | Turbid | Turbid |
| | mg/mL glycerin, 3 mg/mL metacresol | | |
| 11 | 10 mmol/L NaAC-HAC pH 3.5, 20 mg/mL | Turbid | Turbid |
| | glycerin, 3 mg/mL metacresol | | |
| 12 | 10 mmol/L NaAC-HAC pH 3.5, 25 mg/mL | Turbid | Turbid |
| | glycerin, 3 mg/mL metacresol | | |
| 13 | 10 mmol/L histidine pH 4.0, 40 mg/mL | Transparent and | Transparent and |
| | mannitol, 2 mg/mL phenol | clear | clear |
| 14 | 10 mmol/L histidine pH 4.0, 45 mg/mL | Transparent and | Transparent and |
| | mannitol, 2 mg/mL phenol | clear | clear |
| 15 | 10 mmol/L histidine pH 4.0, 50 mg/mL | Transparent and | Transparent and |
| | mannitol, 2 mg/mL phenol | clear | clear |
| 16 | 10 mmol/L histidine pH 4.0, 55 mg/mL | Transparent and | Transparent and |
| | mannitol, 2 mg/mL phenol | clear | clear |
| 17 | 10 mmol/L histidine pH 4.0, 15 mg/mL | Transparent and | Transparent and |
| | glycerin, 2 mg/mL phenol | clear | clear |
| 18 | 10 mmol/L histidine pH 4.0, 20 mg/mL | Transparent and | Transparent and |
| | glycerin, 2 mg/mL phenol | clear | clear |
| 19 | 10 mmol/L histidine pH 4.0, 25 mg/mL | Transparent and | Transparent and |
| | glycerin, 2 mg/mL phenol | clear | clear |
| 20 | 10 mmol/L NaAC-HAC pH 3.5, 40 mg/mL | Transparent and | Transparent and |
| | mannitol, 9 mg/mL benzyl alcohol | clear | clear |
| 21 | 10 mmol/L NaAC-HAC pH 3.5, 45 mg/mL | Transparent and | Transparent and |
| | mannitol, 9 mg/mL benzyl alcohol | clear | clear |
| 22 | 10 mmol/L NaAC-HAC pH 3.5, 50 mg/mL | Transparent and | Transparent and |
| | mannitol, 9 mg/mL benzyl alcohol | clear | clear |

[symbol]

indicates data missing or illegible when filed

As can be seen from the results in Table 4, metacresol severely influenced the stability of GLP-1, which became turbid immediately after the formulation, whereas phenol and benzyl alcohol were better, as the pharmaceutical composition solutions of GLP-1 maintained in a clear and transparent state.

### EXAMPLE 4

### Influence of the Adjuvant on Physical Stability of GLP-1

TABLE 5
Influence of the adjuvant on physical stability of GLP-1

| Pharmaceutical | Composition (GLP-1 final concentration 2 | Appearance | |
|---|---|---|---|
| composition No. | mg/mL, 10 mmol/L NaAC-HAC pH 3.5) | after preparation | 25° C., 14 days |
| 23 | 40 mg/mL sorbitol, 2 mg/mL phenol | Transparent and | Transparent and |
| | | [symbol] | [symbol] |
| 24 | 45 mg/mL sorbitol, 2 mg/mL phenol | Transparent and | Transparent and |
| | | [symbol] | [symbol] |
| 25 | 50 mg/mL sorbitol, 2 mg/mL phenol | Transparent and | Transparent and |
| | | [symbol] | [symbol] |
| 26 | 5 mg/mL hydroxypropyl-beta-cyclodextrin, | Transparent and | Transparent and |
| | 2 mg/mL phenol | [symbol] | [symbol] |
| 27 | 20 mg/mL hydroxypropyl-beta- | Transparent and | Transparent and |
| | cyclodextrin, 2 mg/mL phenol | [symbol] | [symbol] |
| 28 | 5 mg/mL hydroxypropyl-beta-cyclodextrin | Turbid | Turbid |
| | (HP), 3 mg/mL metacresol | | |
| 29 | 20 mg/mL hydroxypropyl-beta- | Turbid | Turbid |
| | cyclodextrin, 3 mg/mL metacresol | | |
| 30 | 5 mg/mL hydroxypropyl-beta-cyclodextrin, | Transparent and | Transparent and |
| | 9 mg/mL benzyl alcohol | [symbol] | [symbol] |
| 31 | 20 mg/mL hydroxypropyl-beta- | Transparent and | Transparent and |
| | cyclodextrin, 9 mg/mL benzyl alcohol | [symbol] | [symbol] |
| 32 | 0.5 mg/mL carboxyl methyl cellulose | Turbid | Turbid |
| | (CMC), 2 mg/mL phenol | | |
| 33 | 0.5 mg/mL carboxyl methyl cellulose | Turbid | Turbid |
| | (CMC), 3 mg/mL metacresol | | |
| 34 | 0.5 mg/mL carboxyl methyl cellulose | Turbid | Turbid |
| | (CMC), 9 mg/mL benzyl alcohol | | |
| 35 | 2 mg/mL carboxyl methyl cellulose (CMC), | Turbid | Turbid |
| | 2 mg/mL phenol | | |
| 36 | 2 mg/mL carboxyl methyl cellulose (CMC), | Turbid | Turbid |
| | 3 mg/mL metacresol | | |
| 37 | 2 mg/mL carboxyl methyl cellulose (CMC), | Turbid | Turbid |
| | 9 mg/mL benzyl alcohol | | |
| 38 | 0.05 mg/mL heparin sodium, 2 mg/mL | Turbid | Turbid |
| | [symbol] | | |
| 39 | 0.05 mg/mL heparin sodium, 3 mg/mL | Turbid | Turbid |
| | metacresol | | |
| 40 | 0.05 mg/mL heparin sodium, 9 mg/mL | Turbid | Turbid |
| | benzyl alcohol | | |
| 41 | 0.3 mg/mL heparin sodium, 2 mg/mL | Turbid | Turbid |
| | phenol | | |
| 42 | 0.3 mg/mL heparin sodium, 3 mg/mL | Turbid | Turbid |
| | metacresol | | |
| 43 | 0.3 mg/mL heparin sodium, 9 mg/mL | Turbid | Turbid |
| | benzyl alcohol | | |

[symbol]

indicates data missing or illegible when filed

Results in Table 5 show that carboxy methylcellulose and heparin sodium are not suitable as adjuvants of GLP-1.

### EXAMPLE 5

### Influence of the Adjuvant (Additive) on the Stability of GLP-1

Formulation of the Pharmaceutical Composition:

The GLP-1 solution (referred to as a stock solution) that had been replaced into different buffer systems (the buffers had a concentration 2 times that of the final pharmaceutical composition) was diluted with a buffer to 4 mg/mL, and an equal volume of a concentrated stock adjuvant solution with a 2-time concentration was added therein. The solutions were adjusted to pH 3.5-4.0, mixed uniformly, filtered through a 0.22 μm filter membrane, dispensed into 2 mL Penicillin bottles, and placed at different temperatures for investigation. A series of sampling time points were arranged. The samples were firstly observed with the naked eye for appearance. If no evident turbidity or gelatination was occurred, the sample was considered as qualified as for physical stability, and then subjected to HPLC detection to analyze the chemical stability.

The HPLC detection method was performed according to Example 1.

TABLE 6
Influence of composition of the pharmaceutical composition on the stability of GLP-1

| | | | Results of HPLC | | | |
|---|---|---|---|---|---|---|
| | | | analysis 0 day | | 25° C., 14 days | |
| | | | after the | | | Retention |
| | | | preparation | | | of the |
| Pharmaceutical | | | | Peptide | | peptide |
| composition | Composition (GLP-1 final | | Purity | concentration | Purity | content |
| No. | concentration 2 mg/mL) | | (%) | (mg/ml) | (%) | (%) |
| 44 | 10 mmol/L | 45 mg/ml mannitol, 2 mg/ml | 97.97 | 1.96 | Gelatination | |
| | histidine- | phenol, 0.1 mg/ml | | | | |
| | HCl pH | Tween 80 | | | | |
| 45 | 4.0 | 45 mg/ml mannitol, 2 mg/ml | 97.78 | 2.00 | 97.12 | 91.51 |
| | | phenol, 0.5 mg/ml | | | | |
| | | Tween 80 | | | | |
| 46 | | 45 mg/ml mannitol, 2% | 97.62 | 2.00 | 97.72 | 86.55 |
| | | phenol 2 mg/ml Tween | | | | |
| | | 80 | | | | |
| 47 | 10 mmol/L | 45 mg/ml mannitol, 2 mg/ml | 98.24 | 1.97 | 97.99 | 92.39 |
| | NaAC- | phenol 0.1 mg/ml | | | | |
| | HAC, pH | Tween 80 | | | | |
| 48 | 3.5 | 45 mg/ml mannitol, 2 mg/ml | 98.35 | 1.96 | 98.42 | 92.21 |
| | | phenol, 0.5 mg/ml | | | | |
| | | Tween 80 | | | | |
| 49 | | 45 mg/ml mannitol, 2 mg/ml | 98.37 | 2.01 | 97.57 | 91.86 |
| | | phenol, 2 mg/ml | | | | |
| | | Tween 80 | | | | |
| 50 | 10 mmol/L | 45 mg/ml mannitol, 2 mg/ml | 97.88 | 2.01 | 97.72 | 80.37 |
| | NaA-HAC, | phenol, 0.2 mg/ml | | | | |
| | pH 4.0 | PEG 400 | | | | |
| 51 | | 45 mg/ml mannitol, 2 mg/ml | 97.78 | 2.03 | 97.79 | 93.55 |
| | | phenol, 0.5 mg/ml | | | | |
| | | PEG 400 | | | | |
| 52 | | 45 mg/ml mannitol, 2 mg/ml | 97.59 | 2.00 | 97.74 | 89.60 |
| | | phenol, 1 mg/ml | | | | |
| | | PEG 400 | | | | |
| 53 | 10 mmol/L | 45 mg/ml mannitol, 2 mg/ml | 98.28 | 2.06 | 97.19 | 90.07 |
| | histidine- | phenol 0.2, mg/ml | | | | |
| | HCl pH | PEG 400 | | | | |
| 54 | 4.0 | 45 mg/ml mannitol, 2 mg/ml | 98.48 | 2.06 | 97.74 | 90.30 |
| | | phenol, 0.5% | | | | |
| | | PEG 400 | | | | |
| 55 | | 45 mg/ml mannitol, 2 mg/ml | 98.42 | 1.85 | 97.39 | 87.39 |
| | | phenol, 1 mg/ml | | | | |

All pharmaceutical compositions of 10 mmol/L histidine pH 4.0 were placed at 4° C. overnight, and then milkiness appeared. When the pharmaceutical compositions were shifted to room temperature (above 25° C.), they became clear.

### EXAMPLE 6

The formulation method and HPLC detection method of the pharmaceutical compositions were performed according to Example 5.

TABLE 7
Influence of composition of the pharmaceutical composition on the stability of GLP-1

| | | Results of HPLC | | | |
|---|---|---|---|---|---|
| | | analysis (0 day) | | | |
| | | after the preparation | | 25° C., 42 days | |
| | | | Peptide | | Retention of the |
| Pharmaceutical | Composition (GLP-1 final concentration 2 | Purity | concentration | Purity | peptide content |
| composition No. | mg/mL, 10 mmol/L NaAC-HAC pH 3.5) | (%) | (mg/mL) | (%) | (%) |
| 56 | 50 mg/mL mannitol + 2 mg/mL | 99.5 | 2.04 | Gelatination | |
| | phenol + 0.5 mg/mL dextran 20 | | | | |
| 57 | 50 mg/mL mannitol + 2 mg/mL | 100 | 2.01 | Gelatination | |
| | phenol + 1 mg/mL dextran 20 | | | | |
| 58 | 50 mg/mL mannitol + 2 mg/mL | 99.5 | 2.14 | Gelatination | |
| | phenol + 2 mg/mL dextran 20 | | | | |
| 59 | 50 mg/mL mannitol + 2 mg/mL + | 99.6 | 2.11 | Gelatination | |
| | 3 mg/mL dextran 20 | | | | |
| 60 | 50 mg/mL mannitol + 2 mg/mL | 99.4 | 1.95 | 97.8 | 98.5 |
| | phenol + 0.1 mg/mL propylene | | | | |
| 61 | 50 mg/mL mannitol + 2 mg/mL | 99.7 | 1.95 | 97.8 | 98.37 |
| | phenol + 0.4 mg/mL propylene | | | | |
| 62 | 50 mg/mL mannitol + 2 mg/mL | 100 | 2.00 | 97.9 | 97.69 |
| | phenol + 1 mg/mL propylene | | | | |

### EXAMPLE 7

The formulation method and HPLC detection method of the pharmaceutical compositions were performed according to Example 5.

TABLE 8
Influence of the ratio of mannitol to propylene glycol on the stability of GLP-1

| | | Results of HPLC | | | |
|---|---|---|---|---|---|
| | | analysis (0 day) | | | |
| | | after the preparation | | 25° C., 42 days | |
| | | | Peptide | | Retention of the |
| Pharmaceutical | Composition (GLP-1 final concentration 2 | Purity | concentration | Purity | peptide content |
| composition No. | mg/mL, 10 mmol/L NaAC-HAC pH 3.5) | (%) | (mg/mL) | (%) | (%) |
| 63 | 40 mg/mL mannitol + 2.2 mg/mL | 99.7 | 2.01 | 97.0 | 96.65 |
| | phenol | | | | |
| 64 | 40 mg/mL mannitol + 2.2 mg/mL | 99.4 | 1.99 | 97.8 | 97.62 |
| | phenol + 0.1 mg/mL propylene | | | | |
| | glycol | | | | |
| 65 | 38 mg/mL mannitol + 2.2 mg/mL | 99.4 | 1.99 | 97.8 | 96.66 |
| | phenol + 1 mg/mL propylene | | | | |
| | glycol | | | | |
| 66 | 35 mg/mL mannitol + 2.2 mg/mL | 99.5 | 2.00 | 97.5 | 97.93 |
| | phenol + 2 mg/mL propylene | | | | |
| | glycol | | | | |
| 67 | 30 mg/mL mannitol + 2.2 mg/mL | 99.5 | 2.00 | 96.7 | 97.19 |
| | phenol + 5 mg/mL propylene | | | | |
| | glycol | | | | |
| 68 | 22 mg/mL mannitol + 2.2 mg/mL | 99.5 | 2.00 | 97.7 | 97.07 |
| | phenol + 7.5 mg/mL propylene | | | | |
| | glycol | | | | |

As can be seen from results in Table 8, two pharmaceutical compositions, i.e., 3.5% mannitol+0.2% propylene glycol and 4% mannitol+0.01% propylene glycol, had the highest retention of the peptide content at 25 degrees at 42 days.

### EXAMPLE 8

An analogue Em of Exendin-4 had a sequence as follows:

HGEGTFTSDL SKQLEEEAVK LFIEWLKNGG PSSGAPPPR

(1) Preparation of Em

The preparation was performed using a solid phase polypeptide synthesis method, and then purification using a reversed phase C18 column and lyophilization were performed, so as to obtain Em.

(2) Formulation Method of the Pharmaceutical Composition

Lyophilized Em powder was weighed, and dissolved with 2 times of a pH 3.5 NaAC-HAC buffer. In addition, mannitol crystalline powder and metacresol were weighed according to an amount 2 times that in recipe, and dissolved in water. Then, the above two solutions were mixed, stirred uniformly, filtered through a 0.22 μm membrane, and dispensed into Penicillin bottles or carlsberg's flasks.

(3) HPLC Detection Method

An investigation method for chemical stability: a test sample was taken, and analyzed on a C18 column (5.0 μm, 300 Å, φ 4.6×150 mm). An analytical method: mobile phase A was 0.1% trifluoroacetic acid, B was the A phase with 80% acetonitrile added therein, the detection wavelength was 214 nm, 68% A and 32% B were balanced for 4 min, and the sample amount was 20 to 40 μL. Elution was performed for 15 min with 32% to 45% of the B phase and 68% to 55% of the A phase. Peak area was calculated using the normalization method. A standard sample was determined with the same method. The peptide concentration in the sample was calculated by comparison with the standard sample, and compared with the determination result at day 0, to calculate the retention of the peptide content in the sample. A higher retention of the peptide content indicated better chemical stability.

TABLE 9
Influence of composition of the composition on the stability of EM

| | | Results of HPLC | | | |
|---|---|---|---|---|---|
| | | analysis (0 day) | | | |
| | | after the preparation | | 25° C., 42 days | |
| | | | Peptide | | Retention of the |
| Pharmaceutical | Composition (EM final concentration 0.3 | Purity | concentration | Purity | peptide content |
| composition No. | mg/mL, 10 mmol/L NaAC-HAC pH 3.5) | (%) | (mg/mL) | (%) | (%) |
| 69 | 50 mg/mL mannitol + 0.3 | 97.6 | 0.296 | 92.78 | 93.40 |
| | mg/mLEDTA-Na₂ + 2.2 mg/mL | | | | |
| | metacresol | | | | |
| 70 | 50 mg/mL mannitol + 0.2 mg/mL | 97.80 | 0.281 | 93.64 | 96.3 |
| | Tween 80 + 2.2 mg/mL metacresol | | | | |
| 71 | 50 mg/mL mannitol + 2.2 mg/mL | 96.33 | 0.289 | 96.05 | 97.37 |
| | metacresol | | | | |
| 72 | 50 mg/mL mannitol + 0.3 mg/mL | 96.86 | 0.288 | 93.75 | 95.14 |
| | EDTA + 2.2 mg/mL metacresol + | | | | |
| | 0.2 mg/mL Tween 80 | | | | |

As can be seen from results in Table 9, the 50 mg/mL mannitol+2.2 mg/mL metacresol formula had the highest retention of the peptide content as well as the best purity at 25 degrees at 30 days. That is to say, the recipe without the addition of EDTA or Tween 80 had good stability, and the recipe with the addition of EDTA or Tween 80 had poor stability.

### EXAMPLE 9

### Influence of Adjuvants (Additives) on the Long-Term Stabilities of GLP-1

GLP-1 formulations (pH 3.5-4.0) comprising 0.2% phenol, 20 mmol/L NaAC-HAC, and the adjuvants listed in Table 10 were prepared as described in Example 5.

The stabilities of GLP-1 were determined by HPLC using a C18 column. The GLP-1 concentration in the sample was determined by comparison with a standard sample as disclosed in Example 1. The GLP-1 purity was determined by comparing the GLP-1 peak area with the total peak area.

The GLP-1 stability parameter (also referred herein as the retention of the peptide content) was determined by comparing the GLP-1 concentration with the GLP-1 concentration on day 0 after preparation (the initial GLP-1 concentration) using the following formula:

GLP-1 Stability Parameter (%)=GLP-1 Concentration/Initial GLP-1 Concentration×100%

TABLE 10
Long-term stability of GLP-1 in GLP-1 formulations

| | | 0 day | | | | | | | |
|---|---|---|---|---|---|---|---|---|---|
| | | after the | | 2-8° C., | | | 2-8° C., | | |
| Pharmaceutical | Composition (main | preparation | | 12 months | | | 24 months | | |
| composition No. | adjuvants) | A* | B** | A* | B** | C*** | A* | B** | C*** |
| 73 | 4.5% mannitol + | 99.1 | 1.96 | 97.55 | 1.90 | 96.94 | 95.0 | 1.89 | 96.43 |
| | 0.5% PEG400 | | | | | | | | |
| 74 | 5% mannitol + | 99.4 | 1.96 | 98.4 | 1.96 | 100.00 | 96.2 | 1.94 | 98.98 |
| | 0.05% dextran | | | | | | | | |
| 75 | 5% mannitol + 0.2% | 99.8 | 1.96 | 98.45 | 1.96 | 100.00 | 96.2 | 1.95 | 99.49 |
| | dextran | | | | | | | | |
| 76 | 5% mannitol + 0.3% | 99.1 | 1.97 | 98.5 | 1.95 | 98.98 | 96.4 | 1.93 | 97.97 |
| | dextran | | | | | | | | |
| 77 | 5% mannitol + | 99.1 | 1.98 | 98.45 | 1.96 | 98.99 | 96.2 | 1.96 | 98.99 |
| | 0.01% propylene | | | | | | | | |
| | glycol | | | | | | | | |
| 78 | 5% mannitol + 0.1% | 98.7 | 2.00 | 98.45 | 1.98 | 99.00 | 96.45 | 1.96 | 98.00 |
| | propylene glycol | | | | | | | | |
| 79 | 5% mannitol + | 99.2 | 1.97 | 98.05 | 1.97 | 100.00 | 96.45 | 1.95 | 98.99 |
| | 0.25% propylene | | | | | | | | |
| | glycol | | | | | | | | |
| 80 | 4.5% mannitol + | 99.2 | 1.97 | 98.05 | 1.96 | 99.49 | 96.7 | 1.96 | 99.49 |
| | 0.1% propylene | | | | | | | | |
| | glycol | | | | | | | | |
| 81 | 4.5% mannitol + | 99.2 | 1.98 | 98.65 | 1.97 | 99.49 | 96.3 | 1.96 | 98.99 |
| | 0.45% propylene | | | | | | | | |
| | glycol | | | | | | | | |

*A is GLP-1 Purity, also referred to as purity in this disclosure (%)
**B is GLP-1 concentration, also referred to as peptide concentration in this disclosure (mg/mL)
***C is GLP-1 Stability Parameter, also referred to as the retention of the peptide content in this disclosure (%)

As shown in Table 10, GLP-1 showed good stabilities in all GLP-1 formulations tested when kept at 4° C. for 2 years. The GLP-1 purities remained at 95% or higher. Among the tested formulations, GLP-1 formulation No. 73 showed the highest degradation rate of GLP-1: 3.57%; and GLP-1 formulation Nos. 75 and 80 showed the best stability parameter of GLP-1: 99.49%.

1. An aqueous parenteral pharmaceutical composition comprising an insulinotropic peptide, a pharmaceutically acceptable osmotic agent, a pharmaceutically acceptable preservative, a pharmaceutically acceptable dissolution enhancer, and a pharmaceutically acceptable buffer salt solution, wherein:
the aqueous parenteral pharmaceutical composition has a pH of 3.0 to 5.0;
the insulinotropic peptide has a concentration of 0.1 mg/mL to 20 mg/mL;
the insulinotropic peptide is GLP-1, Exendin-4, a GLP-1 analogue, an Exendin-4 analogue, a GLP-1 derivative or an Exendin-4 derivative;
GLP-1 and the GLP-1 analogues have a sequence as follows:
(SEQ ID NO: 1)
6 10 20 30 37
X⁶HX⁸EGTFTSD VSSYLEX²²QAA X²⁶EFIAWLVX³⁴G X³⁶X³⁷
wherein, X⁶ is R or a deletion;
X⁸ is A, G or V;
X²² is G or E;
X²⁶ is K, R, Q or N;
X³⁴ is K, R, Q or N;
X³⁶ is R, R—NH₂, K or K—NH₂;
X³⁷ is G or a deletion; and
Exendin-4 and the Exendin-4 analogue have a sequence as follows:
(SEQ ID NO: 2)
X₁X₂X₃GTX₆X₇X₈X₉X₁₀ SKQX₁₄EEEAVX₂₀ LX₂₂X₂₃X₂₄X₂₅LKNGG
X₃₁X₃₂X₃₃X₃₄X₃₅X₃₆X₃₇X₃₈X₃₉
wherein, X₁ is H, R or Y;
X₂ is S, G, A or T;
X₃ is D or E;
X₆ is F or Y;
X₇ is T, Y or S;
X₈ is S or Y;
X₉ is D or E;
X₁₀ is L or I;
X₁₄ is L, I, V or M;
X₂₀ is R or K;
X₂₂ is F or Y;
X₂₃ is I, V, L or M;
X₂₄ is E or D;
X₂₅ is W, For Y;
X₃₁ is P or a deletion;
X₃₂ is S or a deletion;
X₃₃ is S or a deletion;
X₃₄ is G or a deletion;
X₃₅ is A or a deletion;
X₃₆ is P or a deletion;
X₃₇ is P or a deletion;
X₃₈ is P or a deletion; and
X₃₉ is S, R or a deletion.

2. The aqueous parenteral pharmaceutical composition of claim 1, wherein the GLP-1 derivative or the Exendin-4 derivative refers to GLP-1, Exendin-4, a GLP-1 analogue or an Exendin-4 analogue that has a PEGylation modification or fatty acyl modification on one side chain or C-terminus thereof via or not via a spacer arm.

3. The aqueous parenteral pharmaceutical composition of claim 1, wherein the insulinotropic peptide has a concentration of 1 mg/mL to 5 mg/mL.

4. The aqueous parenteral pharmaceutical composition of claim 1, wherein the isotonic agent is polyol, sodium chloride, sugar or any combinations thereof; wherein, the polyol is mannitol, sorbitol, inositol, xylitol, glycerin, propylene glycol or any combinations thereof; and the sugar is sucrose, trehalose, lactose, fructose, glucose or any combinations thereof.

5. The aqueous parenteral pharmaceutical composition of claim 4, wherein:
the polyol has a concentration of 10 mg/mL to 100 mg/mL;
sodium chloride has a concentration of 1 mg/mL to 30 mg/mL; and
the sugar has a concentration of 10 mg/mL to 100 mg/mL.

6. The aqueous parenteral pharmaceutical composition of claim 1, wherein:
the dissolution enhancer is Tween 20, Tween 40, Tween 80, Span 20, Span 40, Span 80, Poloxamer 188, Pluronic F68, Brij 35, dextran-20, PEG 400, PEG 1000, PEG 1500, PEG 2000, propylene glycol or any combinations thereof; and
the dissolution enhancer has a concentration of 0.01 mg/mL to 10 mg/mL.

7. The aqueous parenteral pharmaceutical composition of claim 1, wherein:
when the insulinotropic peptide is GLP-1, the GLP-1 analogue or the GLP-1 derivative, the preservative is phenol, benzyl alcohol, methyl p-hydroxybenzoate, ethyl p-hydroxybenzoate, propyl p-hydroxybenzoate, butyl p-hydroxybenzoate, chlorobutanol, 2-phenoxyethanol, 2-phenethyl alcohol, benzalkonium chloride (bromide), merthiolate or any combinations thereof;
when the insulinotropic peptide is Exendin-4, the Exendin-4 analogue or the Exendin-4 derivative, the preservative is phenol, metacresol, benzyl alcohol, methyl p-hydroxybenzoate, ethyl p-hydroxybenzoate, propyl p-hydroxybenzoate, butyl p-hydroxybenzoate, chlorobutanol, 2-phenoxyethanol, 2-phenethyl alcohol, benzalkonium chloride (bromide), merthiolate or any combinations thereof; and
the preservative has a concentration of 1 mg/mL to 20 mg/mL.

8. The aqueous parenteral pharmaceutical composition of claim 1, wherein the buffer salt is histidine-hydrochloric acid (histidine-HCl), sodium citrate-citric acid, disodium hydrogen phosphate-citric acid, NaOH-citric acid, sodium acetate-acetic acid (NaAC-HAC), succinate-succinic acid, lactate-lactic acid, glutaminate-glutamic acid, malate-malic acid, benzoate-benzoic acid, tartrate-tartaric acid or glycine-hydrochloric acid (Gly-HCl) or any combinations thereof; and the buffer salt has a concentration of 2 to 200 mmol/L.

9. A method of treating diabetes in a subject comprising administering the aqueous parenteral pharmaceutical composition of claim 1 to the subject.

10. A method of treating adiposis in a subject comprising administering the aqueous parenteral pharmaceutical composition of claim 1 to the subject.

11. A method of treating diabetes in a subject comprising administering the aqueous parenteral pharmaceutical composition of claim 2 to the subject.

12. A method of treating adiposis in a subject comprising administering the aqueous parenteral pharmaceutical composition of claim 2 to the subject.

13. A method of treating diabetes in a subject comprising administering the aqueous parenteral pharmaceutical composition of claim 3 to the subject.

14. A method of treating adiposis in a subject comprising administering the aqueous parenteral pharmaceutical composition of claim 3 to the subject.

15. A method of treating diabetes in a subject comprising administering the aqueous parenteral pharmaceutical composition of claim 4 to the subject.

16. A method of treating adiposis in a subject comprising administering the aqueous parenteral pharmaceutical composition of claim 4 to the subject.

17. A method of treating diabetes in a subject comprising administering the aqueous parenteral pharmaceutical composition of claim 5 to the subject.

18. A method of treating adiposis in a subject comprising administering the aqueous parenteral pharmaceutical composition of claim 5 to the subject.

19. A method of treating diabetes in a subject comprising administering the aqueous parenteral pharmaceutical composition of claim 6 to the subject.

20. A method of treating adiposis in a subject comprising administering the aqueous parenteral pharmaceutical composition of claim 6 to the subject.


SEQUENCE LISTING (4 sequences)

SEQ ID NO: 1 - protein, 32 aa, Artificial Sequence
  Synthetic peptide
XHXEGTFTSDVSSYLEXQAAXEFIAWLVXGXX

SEQ ID NO: 2 - protein, 39 aa, Artificial Sequence
  Synthetic peptide
XXXGTXXXXXSKQXEEEAVXLXXXXLKNGGXXXXXXXXX

SEQ ID NO: 3 - protein, 39 aa, Artificial Sequence
  Synthetic peptide
HGEGTFTSDLSKQMEEEAVRLFIEWLKNGGPSSGAPPPS

SEQ ID NO: 4 - protein, 39 aa, Artificial Sequence
  Synthetic peptide
HGEGTFTSDLSKQLEEEAVKLFIEWLKNGGPSSGAPPPR